(19) United States
(12) Patent Application Publication
(10) Pub. No.: US 2026/0290507 A1
(43) Pub. Date: Sep. 24, 2026

(54) SYSTEMS AND METHODS FOR AUTOMATED ERROR CORRECTION OF BIOLOGICAL SAMPLE MEASUREMENTS USING COVARIATE BIOLOGICAL SAMPLE MEASUREMENTS

(71) Applicant: Alden Scientific, Inc., Cambridge, MA (US)

(72) Inventors: James A. Heywood, Newton, MA (US); Timothy E. Vaughan, Cambridge, MA (US); Kriston L. McGary, Flagstaff, AZ (US); Dean E. Cerrato, Boston, MA (US)

(73) Assignee: Alden Scientific, Inc., Cambridge, MA (US)

(21) Appl. No.: 19/572,153

(22) Filed: Mar. 19, 2026

Related U.S. Application Data
(60) Provisional application No. 63/774,169, filed on Mar. 19, 2025.

(51) Int. Cl. G16B 40/10

(52) U.S. Cl. CPC G16B 40/10

(57) ABSTRACT

A method for improved calibration of multi-omics biological measurement includes obtaining training biological measurement data from at least one multi-omics data source for a plurality of training subjects in a plurality of training block data sets associated with shared experimental conditions. For a particular biological measurement, cross-biological measurement correlations are determined between at least one particular training biological measurement and each out-of-block biological measurement from different training block data sets. A biological measurement error model is trained to output at least one predicted biological measurement value and is utilized to predict new predicted biological measurement values for new samples. A block-specific bias offset is determined by statistically aggregating differences between the predicted biological measurement values and measured biological measurement values, and the block-specific bias offset is applied to generate bias-reduced biological measurement values.

### RELATED APPLICATION(S)

This application claims priority to and the benefit of U.S. Provisional Application No. 63/774,169, filed Mar. 19, 2025, the contents of which is incorporated herein by reference in its entirety.

### FIELD OF TECHNOLOGY

Systems and methods herein related to technical fields of biological measurement and correction of error thereof using covariates, including machine learning-based error correction, to compensate for measurement bias and other sources of error.

### BACKGROUND OF TECHNOLOGY

Omics is the collective characterization and quantification of sets of biological molecules and the investigation of how those molecules translate into the structure, function, and dynamics of an organism or group of organisms. Examples of fields of omics include genomics, proteomics, metabolomics, metagenomics, phenomics, transcriptomics, among others.

In omics, typically, advance systems capable of measuring microscopic molecules are needed in order to measure and track the behaviors of the molecules in organisms. Such systems can be subject to various sources of error which can affect downstream analysis. Thus, error correction techniques can be used to increase the fidelity of measurements to actuality.

### SUMMARY

In one embodiment, the disclosure includes a method comprising obtaining, by at least one processor, training protein data from at least one proteomics data source, the training protein data comprising a plurality of training protein measurements for a plurality of proteins of a plurality of training subjects in a plurality of training block data sets; determining, by the at least one processor, for a particular protein of the plurality of proteins in the training protein data, cross-protein correlations between at least one particular training protein measurement of the particular protein and each out-of-block protein measurement of a plurality of out-of-block proteins associated with each out-of-block protein, each out-of-block protein being associated with a different training block data set of the plurality of training block data sets from a particular training block data set, wherein the particular protein is associated with the particular training block data set; updating, by the at least one processor, training for a protein error model to output at least one predicted protein measurement value based at least in part on cross-protein correlations for the particular protein, wherein the training comprises inputting each out-of-block protein measurement associated with each out-of-block protein, outputting the at least one predicted protein measurement value, and updating the protein error model based at least in part on an error between the at least one predicted protein measurement value and the at least one particular training protein measurement of the particular protein; and utilizing, by the at least one processor, the protein error model to predict new predicted protein measurement values for the particular protein in new samples based at least in part on each new out-of-block protein measurement associated with each new out-of-block protein of the plurality of out-of-block proteins in the new samples.

In another embodiment, the disclosure includes a method comprising obtaining, by at least one processor, protein data from at least one sample in at least one block data set, the protein data comprising a plurality of protein measurement values associated with a plurality of proteins in the at least one block data set; obtaining, by the at least one processor, out-of-block protein data from at least one other block data set, the out-of-block protein data comprising a plurality of out-of-block protein measurement values associated with a plurality of out-of-block proteins in the at least one other block data set; utilizing, by the at least one processor, a protein error model to output at least one predicted protein measurement value for a particular protein of the plurality of proteins based at least in part on cross-protein correlations between at least one particular training protein measurement of the particular protein and each out-of-block protein measurement of the plurality of out-of-block proteins; determining, by the at least one processor, a protein measurement bias associated with the at least one block data set based at least in part on the at least one predicted protein measurement value and a particular protein measurement value of the plurality of protein measurement values or the protein data, the particular protein measurement value being associated with the particular protein; and correcting, by the at least one processor, the protein data for the at least one block data set based at least in part on the bias. These and other features will be more clearly understood from the following detailed description taken in conjunction with the accompanying drawings and claims.

In some embodiments, the term “protein measurement” may refer to a measured level, quantity, amount or other measurement of an associated protein present in a corresponding sample.

In some embodiments, the term “panel” may refer to a set of proteins grouped according to function, such as functions created based on, e.g., predefined, configurable, derived, or other technique(s).

In some embodiments, the term “block” and/or “block data set” may refer to a set of proteomic measurements that are, or have been, obtained according to one or more common or shared experimental conditions, such as a common reagent batch, assay plate, sample processing protocol, or other condition or any combination thereof. A block may refer toa single experiment (e.g., a batch from an assay plate), a data set from the same reagent lot, a computational unit of measurement grouping, among other groupings based on experimental conditions.

In some embodiments, the term “in-block” refers to measurements obtained within a given block, and may refer to measurements of the same type (e.g., protein abundance, immunoassays, blood platelet counts, nucleotide measurements, etc.), or may be any measurement from within the block.

In some embodiments, the term “out-of-block” refers to measurements obtained from a block different from that of a given measurement, and may refer to measurements of the same type (e.g., protein abundance, immunoassays, blood platelet counts, nucleotide measurements, etc.), or may be any measurement from outside of the block of the given measurement.

In some embodiments, the term “assay” may refer to an investigative (e.g., analytic) procedure for qualitatively assessing or quantitatively measuring the presence, amount, or functional activity of a target entity, such as a protein measurement, block control value, or other analyte, measurand, or target, among others or any combination thereof.

In some embodiments, the term “block control” may refer to error correction, bias correction, or other pre-processing applied to a block and/or block data set.

In some embodiments, the term “sample” may refer to biological materials obtained from living or deceased subjects, and may include, without limitation, biofluids, tissue, cells, ribonucleic acid, deoxyribonucleic acid (DNA), protein lysate, cell-free DAN (cfDNA), circulating tumor DNA (ctDNA), among others or any combination thereof.

In some embodiments, “biofluid” may include, without limitation, blood, bile, bone Marrow Aspirate, breast milk, Cerebral spinal Fluid (CSF), plasma, saliva, serum, sputum, stool, swabs (oral, nasal, vaginal fluids), synovial fluid, urine, etc.

In some embodiments, “tissue” may include, without limitation, samples collected from a specific organ of the subject, such as, e.g., FFPE blocks, fixed tissue, fresh tissue, frozen tissue, slides, tissue microarrays (TMA), etc.

In some embodiments, the term “response variable,” “dependent variable,” “response measurement,” “dependent measurement,” “response protein,” “dependent protein,” or the like, may refer to a variable of interest to a particular study or analysis, such as the protein measurement value(s) for a particular protein of interest.

In some embodiments, the term “covariate” may refer to a variable that affects a response, or dependent, variable, but not of the variable of interest, such as a protein that influences, is influence by, or is otherwise correlated with the protein measurement value(s) for a particular protein of interest.

In some embodiments, the term “bias” may refer to a systematic tendency in which the methods used to measure and/or gather data and generate statistics present an inaccurate, skewed or otherwise incorrect representation of actuality, such as a systematic offset to protein measurements.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a block diagram illustrating the system for automated error correction of biological sample measurements using covariate biological sample measurements in accordance with one or more embodiments of the present disclosure.

FIG. 2 is a schematic block diagram illustrating a system for automated error correction of biological sample measurements using covariate biological sample measurements in accordance with one or more embodiments of the present disclosure.

FIG. 3 illustrates a schematic flow chart diagram of a method for determining correlations between protein measurements and covariate proteins in accordance with one or more embodiments of the present disclosure.

FIG. 4 is a block diagram illustrating the interaction between protein measurements, covariate proteins, and a prediction model with an optimizer in accordance with one or more embodiments of the present disclosure.

FIG. 5 is a block diagram illustrating a system for reducing bias in biological molecule measurement systems in accordance with one or more embodiments of the present disclosure.

### DETAILED DESCRIPTION

Various detailed embodiments of the present disclosure, taken in conjunction with the accompanying FIGS., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

In the field of biological measurement, accurate data collection and analysis are important for understanding the complex interactions and functions of a particular biological measurement within organisms. However, the measurement can be fraught with challenges, including biases and errors introduced during the data collection process. These inaccuracies can significantly impact the reliability of downstream analyses and the application of predictive models across different data sets. For instance, when attempting to apply models trained on one cohort to another, discrepancies in measurement values can lead to inconsistent and unreliable results.

Existing methods for error correction and bias reduction of biological measurement data often struggle to address these issues effectively. Indeed, traditional methods for error and/or bias correction or normalization for biological measurement data often rely on direct numerical comparisons or simple statistical adjustments. These approaches can be inadequate, as they do not account for complex inter-measurement relationships, or the specific biases introduced in a particular sample and/or block.

These problems are relevant to any field of omics, where the detection and correction of errors and/or bias may pose challenges in genomics, proteomics, metabolomics, metagenomics, phenomics, transcriptomics, among others or any combination, and indeed in other biological measurements including biomarkers and physiological and anatomical measurements, among others or any combination thereof. Thus, while, purely for illustration and ease of understanding, the following refers generally to proteomics, inventors have appreciated that the techniques herein may be applied in any field of omics, and indeed in other biological measurement paradigms.

Some workflows adjust measurements of a plate so that average protein abundance is set to zero or a reference value. However, this can obscure true biological differences if the plate's subjects differ systematically. Others may use basic unit scaling or batch-effect corrections that may not account for sample-specific demographic factors or the underlying inter-relationships among measured items (e.g., proteins). Furthermore, these methods may not adequately account for the systematic biases introduced by shared controls within measurement blocks, leading to spurious correlations that skew the data.

Techniques herein provide innovative approaches to data measurement optimization within different proteomics data sets, specifically addressing the challenge of measurement bias in protein data to provide a flexible, multi-stage normalization approach using in-band and/or out-of-band data signals, preserving biological signals while minimizing batch bias. Such techniques can leverage cross-protein correlations to predict protein measurement values, thereby enabling the correction of biases introduced by shared block controls in proteomics measurements. The approach involves training a protein error model using out-of-block protein measurements to predict and adjust protein values, which enhances the accuracy of predictive models when applied to different cohorts, such as the UKB and Aurora cohorts. By systematically reducing the correlation between within-block proteins, this method provides a more reliable framework for applying predictive models across diverse data sets, thus overcoming a significant impediment in the field of proteomics data analysis.

The proteomic data sets may be obtained from one or more proteomics measurement systems, such as immunoassays, antibody-free protein detection (e.g., including Edman degradation, mass spectrometry-based techniques, etc.), separation-based techniques, mass spectrometric immunoassays (MSIA), Stable Isotope Standard Capture with Anti-Peptide Antibodies (SISCAPA), Fluorescence two-dimensional differential gel electrophoresis (2-D DIGE), affinity proteomics, protein detection via bioorthogonal chemistry, among others or any combination thereof.

In one embodiment, the system for obtaining and processing training protein data can be implemented using a distributed computing architecture, where multiple processors are employed to handle large datasets from various proteomics data sources. This setup allows for parallel processing of training protein measurements, enhancing the efficiency and speed of data analysis. In another embodiment, the system may utilize a cloud-based platform to store and process the training protein data, providing scalability and remote accessibility for users. The cloud-based system can dynamically allocate resources based on the computational demands of the protein error model training and prediction tasks. Additionally, the system could incorporate machine learning algorithms that are specifically tailored to handle the distinct characteristics of proteomics data, such as high dimensionality and noise. These algorithms can be designed to automatically adjust their parameters based on the cross-protein correlations identified during the training phase, thereby improving the accuracy of the predicted protein measurement values. Furthermore, the system might include a user interface that allows researchers to visualize the cross-protein correlations and the impact of the in-band adjustments on the protein data, facilitating a deeper understanding of the measurement optimization process. This interface could be customizable, enabling users to select specific proteins or blocks of interest for detailed analysis.

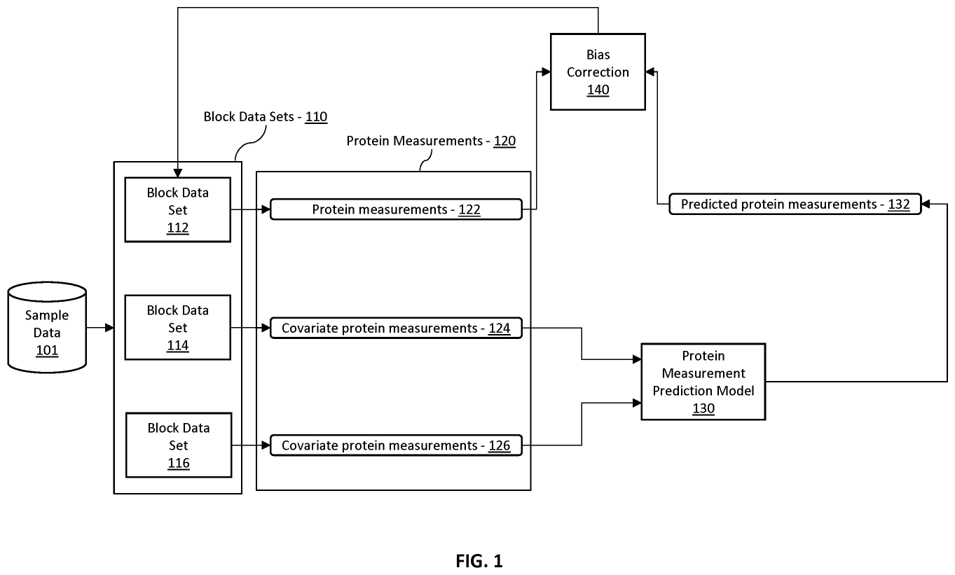

Referring now to FIG. 1, a block diagram is depicted that illustrates the use of a protein measurement prediction model 130 in determining and correcting for bias in protein measurement based on a predicted protein measurement, in accordance with one or more embodiments of the present disclosure.

Proteomics platforms that measure protein amounts and other protein-based biomarkers can have measurement and/or pre-processing biases that can skew values in ways that can impede accuracy as well as comparison and/or measurement optimization with measurements from other systems. Similar issues in measurements can be found in other biologic omics fields, however, for illustrative purposes and ease of understand, embodiments herein are detailed relative to proteomics. The principles detailed herein also apply to other omics.

In some embodiments, in order to address biases, such as measurement bias, internal error control bias, and other sources of bias, bias correction 140 is provided to a block data set 112. Sample data 101, e.g., collected by a proteomics platform such as Olink™ or others, may be grouped in block data sets 110 having protein measurements 120 from one or more samples. The block data sets 110 relate to a set of proteins within a panel that are grouped according to a common or shared block control assay. However, the shared block control assay results in shared bias across proteins in the block data set 110. Thus, bias identified in one or more protein measurements of one or more proteins can be used to correct for bias in the block data set 110.

In some embodiments, the block data sets 110 may include data regarding each sample tested, including the measurement, a date of measurement, a reagent lot, a sample population demographic(s), among other attributes associated with each measurement. The block data sets 110 may include one or more external reference measurements associated with each block, sample, plate, batch, or any combination thereof.

The block data sets 110 may include in-band measurements, such as each measurement associated with sample, and/or out-of-band anchors, such as, e.g., serum CRP, hormone levels (e.g., testosterone, estradiol, etc.), and/or other chemistry panels, anthropometrics and/or demographics (e.g., age, sex, body-mass index (BMI), disease status, medication usage, etc.), omics data (e.g., NMR metabolite panels, methylation-based biomarkers, among other validated molecular data, etc.), among other data or any combination thereof.

In some embodiments, each block data set 110 may be a block or grouping of measurements grouped by one or more factors associated with the measurement of each sample. Examples, of the factor(s) may include, e.g., plate, batch, reagent lot, among others or any combination thereof.

Accordingly, in some embodiments, a protein of interest in a block data set 112 of interest can be identified and used to determine, alone in combination with other proteins of interest, a bias in the block data set 112. To do so, a protein measurement 122 for the protein of interest can be identified and/or extracted. The protein measurement 112 may have a value that is correlated with one or more other proteins. For example, the protein measurement 112 may be influenced by or may influence a protein measurement of a correlated other protein. Thus, the protein measurements of the correlated other protein(s) may have predictive power for the protein of interest.

In some embodiments, the correlated other protein(s) can be identified and their predictive power on the protein of interest can be leveraged, via a protein measurement prediction model 130 to determine an expected protein measurement for the protein of interest. However, correlated other proteins within the same block data set 112 of the protein of interest would be subject to the same block bias as the protein of interest. Thus, correlated other proteins from other block data sets 114 through 116 may be used. Herein, three block data sets 112, 114 and 116 are depicted, though any number of block data sets 110 may be present. By using the correlated proteins from other block data sets 114-116, the bias relative to the aggregate of all block data sets 110 can be determined and used to correct the block data set 112.

Therefore, in some embodiments, the covariate protein measurements 124 through 126 for the correlated other proteins from the other block data sets 114-116 can be used for their predictive power via correlation to the protein of interest. Thus, the covariate protein measurements 124-126 can be input into a protein measurement prediction model 130 trained to use the correlates to the protein of interest in order to produce a predicted protein measurement(s) 132 for the protein of interest.

In some embodiments, the protein measurement prediction model 130 of exemplary inventive computer-based systems and/or devices may be configured to utilize one or more exemplary AI/machine learning techniques chosen from, but not limited to, linear regression models, non-linear regression models, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
a. define Neural Network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the protein measurement prediction model 130 may be specifically tailored to the protein of interest via training on the out-of-block proteins correlated to the protein of interest, e.g., from a training dataset such that the United Kingdom Biobank (UKB). Additionally, or alternatively, one or more models can be trained for multiple proteins of interest, and thus can be reused to make predictions for multiple different proteins.

In some embodiments, because the protein measurement prediction model 130 is trained to predict the predicted protein measurement(s) 132 from protein measurements 124-126 of proteins in block data sets 114-116 different from the block data set 112 of the protein of interest, the bias associated with each block data set 110 can be mitigated. Thus, the predicted protein measurement(s) 132 may be the expected protein measurement for the protein of interest in the absence of bias. Accordingly, in some embodiments, the predicted protein measurement(s) 132 can be used with the protein measurement(s) 122 for the protein of interest for bias correction 140.

In some embodiments, bias correction 140 may be performed based on the difference between the predicted protein measurement(s) 132 and the actual measured protein measurement(s) 122. However, in some embodiments, other errors and biases may be present in a given protein measurement 122 for the protein of interest, e.g., due to additional measurement error, anomalies, artifacts, noise and other sources of error. Thus, the protein measurement(s) 122 and the predicted protein measurement(s) 132 for the protein of interest may be used with the protein measurement(s) 122 and the predicted protein measurement(s) 132 for additional proteins of interest from the same block data set 112. By creating predicted protein measurements 132 from multiple proteins of interest in the block data set 112, the difference between each predicted protein measurement 132 and each actual protein measurement 122 can be statistically aggregated to use variations in errors from across the block data set 112 to cancel each other out. For example, the difference between the predicted protein measurements 132 and the actual protein measurement 122 of each protein from the block data set 112 can be averaged to generate an average bias for protein measurements 122 in the block data set 112. Other forms of statistical aggregation can be used, such as weighted average, etc.

In some embodiments, upon determining the bias, the bias correction 140 can produce an offset to offset the bias in measurement values. As a result, the offset can be applied to the block data set 112 to offset bias in each protein measurement 122. As a result, the correlation of other proteins can be leveraged to determine a likely bias in protein measurements 122 of a particular block data set 112 such that the bias may be removed. In so doing, the unbiased protein measurements 122 can be better optimized, be more accurately and reliable used for proteomics applications, such as new drug discovery, biomarker analysis, diagnostics, among other applications or any combination thereof. For example, the unbiased protein measurements 122 can be leveraged to determine a likely condition, risk or prognosis associated with a patient from which the sample was collected. Such condition, risk or prognosis can be more accurate with the use of unbiased protein measurements.

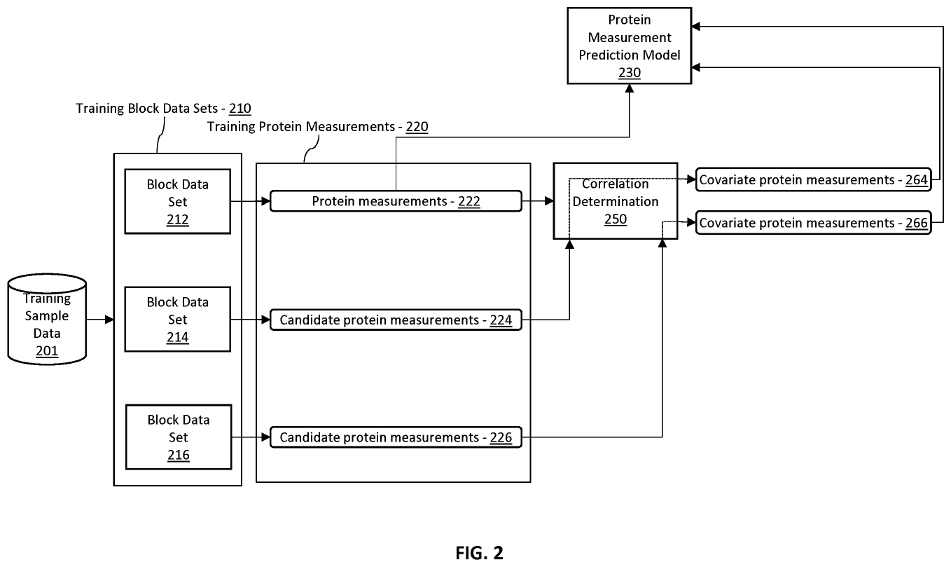

Referring now to FIG. 2, a block diagram is depicted that illustrates the identification of covariate proteins for use in training a protein measurement prediction model 130 to output a predicted protein measurement for a particular protein of interest, in accordance with one or more embodiments of the present disclosure.

In some embodiments, a protein measurement prediction model 230 can be trained on training sample data 230 having training block data sets 210 of training protein measurements 220. Correlations between a protein measurement 222 and potential covariate protein measurements 224 and 226 can be determined and used to build a model that leverages the correlations to infer an expected protein measurement. Accordingly, techniques herein include obtaining, from a training data source, such as, e.g., the UKB or other validated set of biological data (e.g., protein measurements among other biological measurements), training data.

Similar to above, in some embodiments, the training data may be grouped in block data sets 210 having protein measurements 220 from one or more samples. The block data sets 210 relate to a set of proteins within a panel that are grouped according to a common or shared block control assay. However, the shared block control assay results in shared bias across proteins in the block data set 210. Thus, bias identified in one or more protein measurements of one or more proteins can be used to correct for bias in the block data set 210.

Accordingly, in some embodiments, a model can be built for a protein of interest in a block data set 212 of interest. To do so, a protein measurement 222 for the protein of interest can be identified and/or extracted. The protein measurement 212 may have a value that is correlated with one or more other proteins. For example, the protein measurement 212 may be influenced by or may influence a protein measurement of a correlated other protein. Thus, the protein measurements of the correlated other protein(s) may have predictive power for the protein of interest.

Accordingly, in some embodiments, correlation determination 250 may be performed to identify a set of covariate proteins (e.g., correlated proteins) that influence the behavior of the protein of interest. To do so, the correlation determination 250 may obtain candidate protein measurements 224-226 from the proteins in block data sets 214-216 different from the block data set 212 associated with the protein of interest 222.

The candidate protein measurements 224-226 for each candidate protein can be analyzed for a potential correlation to the protein measurement 222 of interest. There is computational cost to the size of a model, and the more proteins used as covariate proteins can increase the size of the model. Thus, there is a balance between the added predictive power of the model due to an additional protein used as a covariate protein, and the computational costs of added the additional protein. Accordingly, using all proteins to model the protein measurement 222 of interest may increase the size of the model without a commensurate benefit in performance or accuracy. Accordingly, the correlation determination 250 may determine a set of, covariate proteins that maximize the performance of the model within a size constraint. For example, the correlation determination 250 may identify the top most correlation candidate proteins to the protein of interest. For example, the top most correlated candidate proteins may be, e.g., a top 10, 20, 30, 40, 50, 60, 70, 80, 90, 100, 150, 200, 250, 300, 350, 400, 450, 500 or more candidate proteins. In another example, the top most correlated candidate proteins may be, e.g., a top 75th percentile, 80th percentile, 85 percentile, 90th percentile, 95th percentile, 96th percentile, 97th percentile, 98th percentile, 99th percentile, or other percentile of candidate proteins.

In some embodiments, to select the covariate proteins, the correlation determination 250 may calculate the correlation coefficients between the protein of interest and each of candidate covariate protein.

The correlation coefficient is a measure that quantifies the strength of the linear relationship between two variables in a correlation analysis. For two variables, the formula compares the distance of each datapoint from the variable mean and uses this to explain how closely the relationship between the variables can be fit to a linear relationship describing the data. For example, the formula used be, e.g., Pearson correlation, Intraclass Correlation, Spearman's rank, Kendall tau rank, Goodman and Kruskal's gamma, among others or any combination thereof.

For example, in some embodiments, the correlation determination 250 may iterate through each candidate protein in each other training block data set 214-216 to assess the correlation between the corresponding corpus of candidate protein measurements 224-226 data of each candidate protein to the protein measurements 222 of the protein of interest. After the correlation coefficient of each candidate protein is determined, the correlation determination 250 may select the most correlated candidate proteins, and output the associated covariate protein measurements 264-266.

Using the covariate protein measurements 264-266, a model, such as a linear or non-linear model may be built that models the protein measurements 222 as a function of the covariate protein measurements 264-266. As a result, the protein measurement prediction model 230 may be trained to enable prediction of an expected protein measurement for the protein of interest based on the protein measurements of out-of-block covariate proteins. In some embodiments, a separate model may be built for each protein in the training sample data 201.

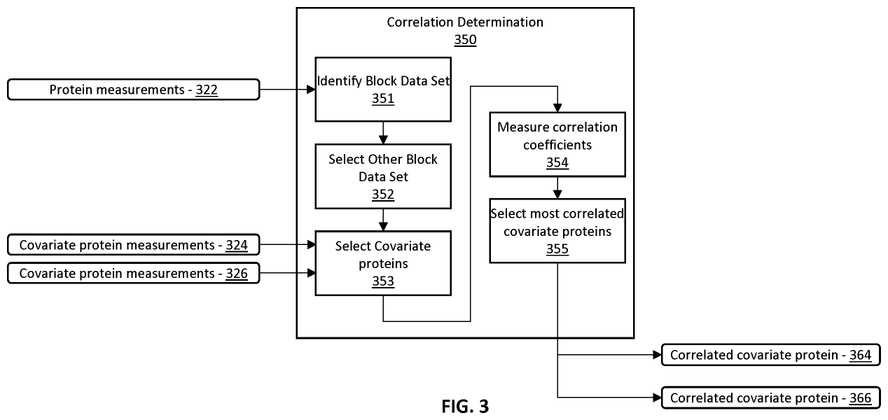

Referring now to FIG. 3, another block diagram is depicted that illustrates the identification of covariate proteins that are correlated to protein measurement values of a particular protein of interest, in accordance with one or more embodiments of the present disclosure.

The Protein Measurements 322 represent the actual measured values of a protein of interest within a specific block, which are subject to biases introduced by shared block controls. These measurements serve as the baseline data from which correlations with covariate proteins are assessed. The protein measurements are important for identifying systematic biases and for training models that predict expected protein values when such biases are not present.

The Covariate Protein Measurements 324 and 326 may include measurements taken from proteins that are not within the same block as the protein of interest, thus providing an external reference that is not subject to the same block-specific biases. By analyzing these covariate protein measurements, the system can identify proteins that have a predictive relationship with the protein of interest. These relationships are leveraged to adjust the protein measurements, thereby reducing the impact of block-specific biases. The selection of appropriate covariate proteins is important, as this choice influences the accuracy of the predicted protein measurements.

The Correlation Determination 350 component is a mechanism that identifies and quantifies the relationships between the protein of interest and potential covariate proteins. This component involves several sub-processes, including the identification of the block data set containing the protein of interest 351, the selection of other block data sets for potential covariate proteins 352, and the selection of covariate proteins 353. The correlation determination process measures correlation coefficients 354 to assess the strength of the relationships between the protein of interest and candidate covariate proteins. The most correlated covariate proteins are then selected 355 for use in the prediction model. This process can ensure that the prediction model is based on the most relevant and informative data.

The Correlated Covariate Proteins 364 and 366 components represent the proteins identified as having a significant correlation with the protein of interest. These proteins are selected based on their correlation coefficients, which indicate the strength and direction of their relationship with the protein of interest. The correlated covariate proteins are used as inputs to the protein measurement prediction model, providing the data needed to predict the expected protein measurement values. The selection of these proteins can influence the effectiveness of the bias correction and the accuracy of the predicted protein measurements.

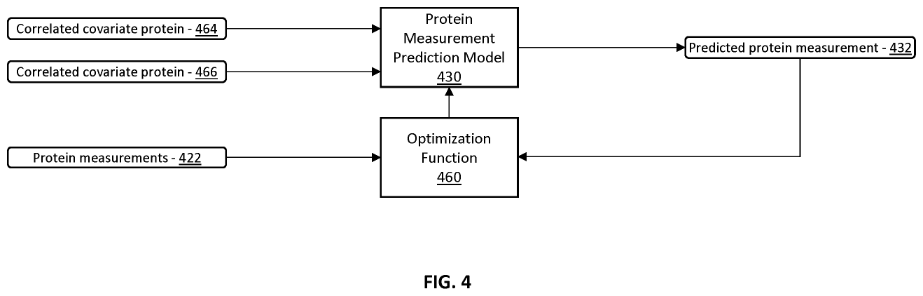

Referring to FIG. 4, a block diagram is depicted that illustrates the interaction between protein measurements, covariate proteins, and a prediction model with an optimizer in accordance with one or more embodiments of the present disclosure.

In some embodiments, the protein measurement prediction model 430 to predict a predicted protein measurement 432 indicated of the value predicted for a particular protein of interest. In some embodiments, the protein measurement prediction model 430 ingests a feature vector that encodes features representative of protein measurement values of correlated covariate proteins 464, 466, e.g., as detailed above with reference to FIG. 3. In some embodiments, the protein measurement prediction model 430 processes the feature vector with parameters to produces a prediction of predicted protein measurement 432. In some embodiments, the parameters of the protein measurement prediction model 430 may be implemented in a suitable machine learning model including a prediction machine learning model, such as, e.g., Linear Regression, Logistic Regression, Ridge Regression, Lasso Regression, Polynomial Regression, Bayesian Linear Regression (e.g., Naive Bayes regression), a convolutional neural network (CNN), a recurrent neural network (RNN), decision trees, random forest, support vector machine (SVM), K-Nearest Neighbors, or any other suitable algorithm for predicting output values based on input values. In some embodiments, for computational efficiency while preserving accuracy of predictions, the protein measurement prediction model 430 may advantageously include a random forest model.

In some embodiments, the protein measurement prediction model 430 processes the features encoded in the feature vector by applying the parameters of the prediction machine learning model to produce a model output vector. In some embodiments, the model output vector may be decoded to generate one or more numerical output values indicative of predicted protein measurement 432. In some embodiments, the model output vector may include or may be decoded to reveal the output value(s) based on a modelled correlation between the feature vector and a target output. In some embodiments, the numerical output may represent the protein measurement value(s) for the predicted protein measurement 432 for the particular protein of interest.

In some embodiments, the parameters of the protein measurement prediction model 430 may be trained based on known outputs. For example, the protein measurement values for correlated covariate proteins 464, 466 may be paired with a target value or known value to form a training pair, such as a historical protein measurement values for correlated covariate proteins 464, 466 and an observed result and/or human annotated value representing a data point in the relationship between the historical protein measurement values for correlated covariate proteins 464, 466 and the predicted protein measurement 432 for the particular protein of interest. In some embodiments, the protein measurement values for correlated covariate proteins 464, 466 may be provided to the protein measurement prediction model 430, e.g., encoded in a feature vector, to produce a predicted output value. In some embodiments, an optimization function 460 associated with the protein measurement prediction model 430 may then compare the predicted output value with the known output of a training pair including the historical protein measurement values for correlated covariate proteins 464, 466 to determine an error of the predicted output value. In some embodiments, the optimization function 460 may employ a loss function, such as, e.g., Hinge Loss, Multi-class SVM Loss, Cross Entropy Loss, Negative Log Likelihood, or other suitable classification loss function to determine the error of the predicted output value based on the known output.

In some embodiments, the known output may be obtained after the protein measurement prediction model 430 produces the prediction, such as in online learning scenarios. In such a scenario, the protein measurement prediction model 430 may receive the protein measurement values for correlated covariate proteins 464, 466 and generate the model output vector to produce an output value representing the protein measurement value(s) for the predicted protein measurement 432 for the particular protein of interest. Subsequently, a user may provide feedback by, e.g., modifying, adjusting, removing, and/or verifying the output value via a suitable feedback mechanism, such as a user interface device (e.g., keyboard, mouse, touch screen, user interface, or other interface mechanism of a user device or any suitable combination thereof). The feedback may be paired with the protein measurement values for correlated covariate proteins 464, 466 to form the training pair and the optimization function 460 may determine an error of the predicted output value using the feedback.

In some embodiments, based on the error, the optimization function 460 may update the parameters of the protein measurement prediction model 430 using a suitable training algorithm such as, e.g., backpropagation for a prediction machine learning model. In some embodiments, backpropagation may include any suitable minimization algorithm such as a gradient method of the loss function with respect to the weights of the prediction machine learning model. Examples of suitable gradient methods include, e.g., stochastic gradient descent, batch gradient descent, mini-batch gradient descent, or other suitable gradient descent technique. As a result, the optimization function 460 may update the parameters of the protein measurement prediction model 430 based on the error of predicted labels in order to train the protein measurement prediction model 430 to model the correlation between protein measurement values for correlated covariate proteins 464, 466 and predicted protein measurement 432 in order to produce more accurate output values based on protein measurement values for correlated covariate proteins 464, 466.

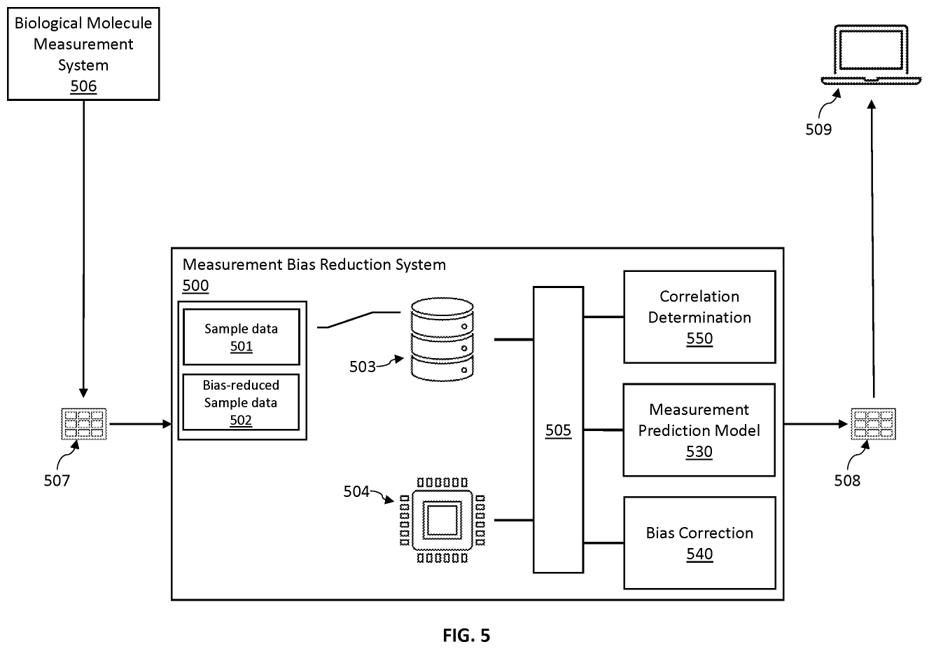

Referring now to FIG. 5, a block diagram is depicted illustrating a system for reducing bias in biological molecule measurement systems in accordance with one or more embodiments of the present disclosure.

In some embodiments, a measurement bias reduction system 500 may curate, collect, obtain, access or otherwise use sample data 501 from biological measurements 507 collected by a biological molecule measurement system 506, such as a proteomics, genomics, or other omics measurement and/or analysis system. The sample data 501 may be stored in a database 503 and processed to produce bias-reduced sample data 502.

In some embodiments, the measurement bias reduction system 500 may include hardware components such as a processor(s) 504, which may include local or remote processing components. In some embodiments, the processor(s) 504 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor(s) 504 may include data-processing capacity provided by the microprocessor. In some embodiments, the microprocessor may include memory, processing, interface resources, controllers, and counters. In some embodiments, the microprocessor may also include one or more programs stored in memory.

Similarly, the measurement bias reduction system 500 may include storage 503, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage 503 may include, e.g., a suitable non-transient computer readable medium such as, e.g., random access memory (RAM), read only memory (ROM), one or more buffers and/or caches, among other memory devices or any combination thereof.

In some embodiments, the measurement bias reduction system 500 may implement computer engines for correlation determination 550, a measurement prediction model 530, and/or bias correction 540. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to determine covariate molecules and/or measurements, the measurement bias reduction system 500 may include computer engines for the correlation determination 550, such as for the correlation determination 250/350 detailed above. In some embodiments, the computer engines for the correlation determination 550 may include dedicated and/or shared software components, hardware components, or a combination thereof. For example, the computer engines for the correlation determination 550 may include a dedicated processor and storage. However, in some embodiments, the computer engines for the correlation determination 550 may share hardware resources, including the processor(s) 504 and storage 503 of the measurement bias reduction system 500 via, e.g., a bus 505.

In some embodiments, to determine expected or predicted measurements, the measurement bias reduction system 500 may include computer engines for the measurement prediction model 530, such as for the measurement prediction model 130, 230 and/or 430 detailed above. In some embodiments, the computer engines for the correlation determination 550 may include dedicated and/or shared software components, hardware components, or a combination thereof. For example, the computer engines for the correlation determination 550 may include a dedicated processor and storage. However, in some embodiments, the computer engines for the correlation determination 550 may share hardware resources, including the processor(s) 504 and storage 503 of the measurement bias reduction system 500 via, e.g., a bus 505.

In some embodiments, to use expected or predicted measurements for bias reduction, the measurement bias reduction system 500 may include computer engines for the bias correction 540, such as for the bias correction 140 detailed above. In some embodiments, the computer engines for the correlation determination 550 may include dedicated and/or shared software components, hardware components, or a combination thereof. For example, the computer engines for the correlation determination 550 may include a dedicated processor and storage. However, in some embodiments, the computer engines for the correlation determination 550 may share hardware resources, including the processor(s) 504 and storage 503 of the measurement bias reduction system 500 via, e.g., a bus 505.

In some embodiments, the measurement bias reduction system 500 may output bias-reduced biological measurements 508 for the biological measurements 507. The bias-reduced biological measurements 508 may be output to a computing system 509 associated with a clinical or other user that associated with the biological measurements 507.

In some embodiments, the user may be a clinician utilizing clinical decision support software of the computing system 509. In some embodiments, clinical decision support software (CDSS) may be a health information technology that provides clinicians, staff, patients, and other individuals with knowledge and person-specific information to help health and health care to enhance decision-making in the clinical workflow. The CDSS may include computerized alerts and reminders to care providers and patients, clinical guidelines, condition-specific order sets, focused patient data reports and summaries, documentation templates, diagnostic support, and contextually relevant reference information, among other tools.

In some embodiments, the CDSS of the computing system 509 may include a knowledge-based system, including, without limitation a knowledge base of protein measurements and/or proteomic data, an inference engine, and a mechanism to communicate. The knowledge base may include the rules and associations of protein measurements and/or proteomic data, including, without limitation, IF-THEN rules. The inference engine may combine the rules from the knowledge base with the bias-reduced biological measurements 508 to produce clinical support recommendations, such as recommended diagnoses, triage recommendations, study design recommendations, among other clinical support tasks or any combination thereof.

In some embodiments, the CDSS may include non-knowledge-based CDSS which may not use a knowledge base, but may instead use machine learning or other artificial intelligence. Machine learning (ML) based CDSS may be used to generate similar clinical decision support, but since systems based on ML may not explain the reasons for their conclusions, the ML may instead be employed as post-diagnostic systems, for suggesting patterns for clinicians to look into in more depth.

In some embodiments, the ML-based CDSS may include one or more AI and/or ML techniques, including those detailed further above, such as support-vector machines, artificial neural networks and genetic algorithms, among others or any combination thereof. For example, genetic algorithms, which are based on simplified evolutionary processes using directed selection to achieve optimal CDSS results, may use the selection algorithms to evaluate components of random sets of solutions to a problem based on the bias-reduced biological measurements 508. The solutions that come out on top may then be recombined and mutated and run through the process again until the proper solution is discovered.

In some embodiments, the computing system 509 may include software and/or systems for regulatory-compliant reporting. In some embodiments, the computing system 509 may update electronic health records (HER), insurance claims, drug study data, health study data, among other data records, with the bias-reduced biological measurements 508. In some embodiments, the computing system 509 may include both the original biological measurements 507 as well as the bias-reduced biological measurements 508 for auditability. As a result, the computing system 509 may be used to report data, including the bias-reduced biological measurements 508, compliant with FDA, CLIA, or other regulatory standard or any combination thereof.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999) , at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

Clause 1. A method comprising: obtaining, by the at least one processor, training biological measurement data from at least one multi-omics data source, the training biological measurement data comprising a plurality of training biological measurement measurements for plurality of biological measurements of a plurality of training subjects in a plurality of training block data sets; determining, by the at least one processor, for a particular biological measurement of the plurality of biological measurements in the training biological measurement data, cross-biological measurement correlations between: at least one particular training biological measurement of the particular biological measurement and each out-of-block biological measurement of a plurality of out-of-block biological measurements associated with each out-of-block biological measurement, each out-of-block biological measurement being associated with a different training block data set of the plurality of training block data sets from a particular training block data set; wherein the particular biological measurement is associated with the particular training block data set; updating, by the at least one processor, training for a biological measurement error model to output at least one predicted biological measurement value based at least in part on cross-biological measurement correlations for the particular biological measurement, wherein the training comprises: inputting each out-of-block biological measurement associated with each out-of-block biological measurement; outputting the at least one predicted biological measurement value; and updating the biological measurement error model based at least in part on an error between the at least one predicted biological measurement value and the at least one particular training biological measurement of the particular biological measurement; utilizing, by the at least one processor, the biological measurement error model to predict new predicted biological measurement values for the particular biological measurement in new samples based at least in part on each new out-of-block biological measurement associated with each new out-of-block biological measurement of the plurality of out-of-block biological measurements in the new samples; and correcting, by the at least one processor, an error in new particular biological measurement values associated with the particular biological measurement in the new samples based at least in part on the new predicted biological measurement values.

Clause 2. The method of clause 1, wherein the at least one multi-omics data source comprises at least one proteomics measurement method.

Clause 3. The method of clause 2, wherein the at least one proteomics measurement method comprises at least one of: at least one immunoassay, at least one mass spectrometry, or any others or any combination thereof.

Clause 4. The method of clause 1, wherein the particular biological measurement comprises a protein abundance measurement.

Clause 5. The method of clause 1, further comprising clinical decision support software configured to: determine, upon correcting the error, at least one diagnosis recommendation based at least in part on the new particular biological measurement values.

Clause 6. The method of clause 5, wherein the clinical decision support software is further configured to: enter the at least one diagnosis recommendation into an electronic health record of a patient associated with the new samples.

Clause 7. The method of clause 5, wherein the clinical decision support software is further configured to: render, on a display of a computing device associated with a clinician, at least one notification representing the at least one diagnosis recommendation.

Clause 8. A method comprising: obtaining, by at least one processor, protein data from at least one sample in at least one block data set, the protein data comprising a plurality of protein measurement values associated with a plurality of proteins in the at least one block data set; obtaining, by the at least one processor, an out-of-block protein data from at least one other block data set, the out-of-block protein data comprising a plurality of out-of-block protein measurement values associated with a plurality of out-of-block proteins in the at least one other block data set; utilizing, by the at least one processor, a protein error model to output at least one predicted protein measurement value for a particular protein of the plurality of proteins based at least in part on cross-protein correlations between: at least one particular training protein measurement of the particular protein and each out-of-block protein measurement of the plurality of out-of-block proteins; determining, by the at least one processor, a protein measurement bias associated with the at least one block data set based at least in part on: the at least one predicted protein measurement value, and a particular protein measurement value of the plurality of protein measurement values or the protein data, the particular protein measurement value being associated with the particular protein; and correcting, by the at least one processor, the protein data for the at least one block data set based at least in part on the bias.

Clause 9. The method of clause 8, wherein the at least one multi-omics data source comprises at least one proteomics measurement method.

Clause 10. The method of clause 9, wherein the at least one proteomics measurement method comprises at least one of: at least one immunoassay, at least one mass spectrometry, or any others or any combination thereof.

Clause 11. The method of clause 8, wherein the particular biological measurement comprises a protein abundance measurement.

Clause 12. The method of clause 8, further comprising clinical decision support software configured to: determine, upon correcting the error, at least one diagnosis recommendation based at least in part on the new particular biological measurement values.

Clause 13. The method of clause 12, wherein the clinical decision support software is further configured to: enter the at least one diagnosis recommendation into an electronic health record of a patient associated with the new samples.

Clause 14. The method of clause 12, wherein the clinical decision support software is further configured to: render, on a display of a computing device associated with a clinician, at least one notification representing the at least one diagnosis recommendation.

The aforementioned examples are, of course, illustrative and not restrictive.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method for improved calibration of multi-omics biological measurement, the method comprising:
obtaining, by the at least one processor, training biological measurement data from at least one multi-omics data source, the training biological measurement data comprising a plurality of training biological measurement measurements for plurality of biological measurements of a plurality of training subjects in a plurality of training block data sets, each training block data set being associated with one or more shared experimental conditions;
determining, by the at least one processor, for a particular biological measurement of the plurality of biological measurements in the training biological measurement data, cross-biological measurement correlations between:
at least one particular training biological measurement of the particular biological measurement and
each out-of-block biological measurement of a plurality of out-of-block biological measurements associated with each out-of-block biological measurement, each out-of-block biological measurement being associated with a different training block data set of the plurality of training block data sets from a particular training block data set;
wherein the particular biological measurement is associated with the particular training block data set;
updating, by the at least one processor, training for a biological measurement error model to output at least one predicted biological measurement value based at least in part on cross-biological measurement correlations for the particular biological measurement, wherein the training comprises:
inputting each out-of-block biological measurement associated with each out-of-block biological measurement;
outputting the at least one predicted biological measurement value; and
updating the biological measurement error model based at least in part on an error between the at least one predicted biological measurement value and the at least one particular training biological measurement of the particular biological measurement;
utilizing, by the at least one processor, the biological measurement error model to predict new predicted biological measurement values for the particular biological measurement in new samples based at least in part on each new out-of-block biological measurement associated with each new out-of-block biological measurement of the plurality of out-of-block biological measurements in the new samples; and
determining, by the at least one processor, a block-specific bias offset for the new block data set by statistically aggregating differences between the predicted biological measurement values and measured biological measurement values for the plurality of biological measurements in the new block data set; and
applying, by the at least one processor, the block-specific bias offset to the measured biological measurement values in the new block data set to generate bias-reduced biological measurement values.

2. The method of claim 1, wherein the at least one multi-omics data source comprises at least one proteomics measurement method.

3. The method of claim 2, wherein the at least one proteomics measurement method comprises at least one of: at least one immunoassay, at least one mass spectrometry, or any others or any combination thereof.

4. The method of claim 1, wherein the particular biological measurement comprises a protein abundance measurement.

5. The method of claim 1, further comprising clinical decision support software configured to: determine, upon correcting the error, at least one diagnosis recommendation based at least in part on the new particular biological measurement values.

6. The method of claim 5, wherein the clinical decision support software is further configured to: enter the at least one diagnosis recommendation into an electronic health record of a patient associated with the new samples.

7. The method of claim 5, wherein the clinical decision support software is further configured to:
render, on a display of a computing device associated with a clinician, at least one notification representing the at least one diagnosis recommendation.

8. A method for improved calibration of multi-omics biological measurement, the method comprising:
obtaining, by at least one processor, protein data from at least one sample in at least one block data set, the protein data comprising a plurality of protein measurement values associated with a plurality of proteins in the at least one block data set;
obtaining, by the at least one processor, an out-of-block protein data from at least one other block data set, the out-of-block protein data comprising a plurality of out-of-block protein measurement values associated with a plurality of out-of-block proteins in the at least one other block data set;
utilizing, by the at least one processor, a protein error model to output at least one predicted protein measurement value for a particular protein of the plurality of proteins based at least in part on cross-protein correlations between:
at least one particular training protein measurement of the particular protein and
each out-of-block protein measurement of the plurality of out-of-block proteins;
determining, by the at least one processor, a protein measurement bias associated with the at least one block data set based at least in part on:
the at least one predicted protein measurement value, and
a particular protein measurement value of the plurality of protein measurement values or the protein data, the particular protein measurement value being associated with the particular protein; and
determining, by the at least one processor, a block-specific protein measurement bias for the block data set by statistically aggregating differences between the predicted protein measurement values and measured protein measurement values for the plurality of proteins in the block data set; and
correcting, by the at least one processor, the protein data for the block data set by applying the block-specific protein measurement bias to the protein measurement values to generate bias-reduced protein measurement values.

9. The method of claim 8, wherein the at least one multi-omics data source comprises at least one proteomics measurement method.

10. The method of claim 9, wherein the at least one proteomics measurement method comprises at least one of: at least one immunoassay, at least one mass spectrometry, or any others or any combination thereof.

11. The method of claim 8, wherein the particular biological measurement comprises a protein abundance measurement.

12. The method of claim 8, further comprising clinical decision support software configured to:
determine, upon correcting the error, at least one diagnosis recommendation based at least in part on the new particular biological measurement values.

13. The method of claim 12, wherein the clinical decision support software is further configured to: enter the at least one diagnosis recommendation into an electronic health record of a patient associated with the new samples.

14. The method of claim 12, wherein the clinical decision support software is further configured to: render, on a display of a computing device associated with a clinician, at least one notification representing the at least one diagnosis recommendation.

15. A system for improved calibration of multi-omics biological measurement, the system comprising:
at least one storage configured to store biological measurement data comprising a plurality of biological measurement values associated with a plurality of biological measurements in a block data set and a plurality of out-of-block biological measurement values associated with a plurality of out-of-block biological measurements in at least one other block data set; and
at least one processor configured to:
obtain training biological measurement data from at least one multi-omics data source, the training biological measurement data comprising a plurality of training biological measurement measurements for plurality of biological measurements of a plurality of training subjects in a plurality of training block data sets;
determine for a particular biological measurement of the plurality of biological measurements in the training biological measurement data, cross-biological measurement correlations between:
at least one particular training biological measurement of the particular biological measurement and
each out-of-block biological measurement of a plurality of out-of-block biological measurements associated with each out-of-block biological measurement, each out-of-block biological measurement being associated with a different training block data set of the plurality of training block data sets from a particular training block data set;
wherein the particular biological measurement is associated with the particular training block data set;
update training for a biological measurement error model to output at least one predicted biological measurement value based at least in part on cross-biological measurement correlations for the particular biological measurement, wherein the training comprises:
inputting each out-of-block biological measurement associated with each out-of-block biological measurement;
outputting the at least one predicted biological measurement value; and
updating the biological measurement error model based at least in part on an error between the at least one predicted biological measurement value and the at least one particular training biological measurement of the particular biological measurement;
utilize the biological measurement error model to predict new predicted biological measurement values for the particular biological measurement in new samples based at least in part on each new out-of-block biological measurement associated with each new out-of-block biological measurement of the plurality of out-of-block biological measurements in the new samples; and
determine a block-specific bias offset for the block data set by statistically aggregating differences between the predicted biological measurement values and measured biological measurement values for the plurality of biological measurements in the block data set;
apply the block-specific bias offset to the original biological measurement values to generate bias-reduced biological measurement values;
store the bias-reduced biological measurement values in the at least one storage; and
output the bias-reduced biological measurement values to a clinical computing system.

16. The system of claim 15, further comprising clinical decision support software configured to determine, upon correcting the error, at least one diagnosis recommendation based at least in part on the new particular biological measurement values.

17. The system of claim 15, wherein the at least one multi-omics data source comprises at least one proteomics measurement method.

18. The system of claim 17, wherein the at least one proteomics measurement method comprises at least one immunoassay or at least one mass spectrometry.

19. The system of claim 15, wherein the particular biological measurement comprises a protein abundance measurement.

20. The system of claim 15, wherein the block data set comprises a set of biological measurements obtained according to one or more common experimental conditions comprising a common reagent batch, assay plate, or sample processing protocol.
